(19) United States
(12) Patent Application Publication
(10) Pub. No.: US 2022/0051779 A1
(43) Pub. Date: Feb. 17, 2022

(54) DYNAMICALLY ALTERING AN EXTERNAL GEOMETRY OF BODY-WEARABLE ACTUATABLE COMPONENTS

(71) Applicant: Amazon Technologies, Inc., Seattle, WA (US)

(72) Inventors: Beth A. MARCUS, Bedford, MA (US); Theodore Joseph COLLINS, Castle Pines, CO (US); Phil CHURCHILL, Hubbardston, MA (US); Matthew Eli CARNEY, Somerville, MA (US)

(21) Appl. No.: 17/113,685

(22) Filed: Dec. 7, 2020

Related U.S. Application Data
(63) Continuation of application No. 63/064,474, filed on Aug. 12, 2020.

(51) Int. Cl. G16H 20/30; G06N 20/00; A61F 5/01; A61F 5/14; A61B 5/11; A61F 5/32; A61B 5/00

(52) U.S. Cl. CPC G16H 20/30; G06N 20/00; A61F 5/0109; A61B 5/7264; A61B 5/112; A61F 5/32; A61F 5/14

(57) ABSTRACT

Aspects described herein include a method comprising receiving one or more sensor signals from one or more sensors that are worn by a wearer, and determining, based on the one or more sensor signals, one or more poses of a leg of the wearer during motion. The method further comprises controlling, based on the one or more poses, a pressure of one or more actuatable components to adjust the leg toward a target pose during the motion. The one or more actuatable components are worn on one or both of the leg and a foot of the wearer.

### CROSS-REFERENCE TO RELATED APPLICATIONS

This application claims benefit to U.S. provisional patent application Ser. No. 63/064,474, filed Aug. 12, 2020. The aforementioned patent application is herein incorporated by reference in its entirety.

### BACKGROUND

The present disclosure relates to body-wearable actuatable components, and more specifically, to dynamically altering an external geometry of body-wearable actuatable components during motion of the wearer.

### BRIEF DESCRIPTION OF DRAWINGS

FIG. 1 illustrates an exemplary environment for dynamically altering an external geometry of body-wearable actuatable components during gait cycles, according to one or more embodiments.

FIG. 2A is a system of dynamically altering an external geometry of body-wearable actuatable components during gait cycles, according to one or more embodiments.

FIG. 2B is a system of dynamically altering an external geometry of foot-wearable actuatable components during gait cycles, according to one or more embodiments.

FIG. 3A illustrates a foot-wearable apparatus with independent inflatable bladders, according to one or more embodiments.

FIG. 3B illustrates a foot-wearable apparatus with inflatable bladders in fluid communication, according to one or more embodiments.

FIG. 3C illustrates a longitudinal arch support having an inflatable bladder, according to one or more embodiments.

FIG. 4A illustrates a foot-wearable apparatus having a heel support with a rotational actuator, according to one or more embodiments.

FIG. 4B illustrates a rotational actuator in a retracted configuration, according to one or more embodiments.

FIG. 4C illustrates a rotational actuator in an extended configuration, according to one or more embodiments.

FIG. 5A illustrates a perspective view of a knee-wearable apparatus, according to one or more embodiments.

FIG. 5B illustrates a front view of a knee-wearable apparatus, according to one or more embodiments.

FIG. 5C illustrates two inflatable bladders for a knee-wearable apparatus, according to one or more embodiments.

FIG. 6 is a diagram illustrating sensor signals and pressure signals at different phases of gait cycles, according to one or more embodiments.

FIG. 7A illustrates a two-phase state machine, according to one or more embodiments.

FIG. 7B illustrates a four-phase state machine, according to one or more embodiments.

FIG. 8A is a diagram illustrating a control system for an inflatable bladder in a longitudinal arch support, according to one or more embodiments.

FIG. 8B is a diagram illustrating a control system for inflatable bladders in a longitudinal arch support and a heel support, according to one or more embodiments.

FIG. 9 is a method of dynamically altering an external geometry of body-worn actuatable components, according to one or more embodiments.

FIG. 10 is a method of controlling a pressure of body-worn actuatable components to adjust a leg toward a target pose, according to one or more embodiments.

FIG. 11 is a method of controlling a bladder system to alter an external geometry of foot-worn actuatable components, according to one or more embodiments.

FIG. 12 is a diagram illustrating a technique for determining a pose of a leg and foot of a wearer, according to one or more embodiments.

### DETAILED DESCRIPTION

Musculoskeletal disorders are extremely common among adults and impose substantial costs associated with treatment and a reduced quality of life. Some typical areas of chronic pain caused by musculoskeletal disorders include the lower back, knees, neck, and shoulders. Techniques described herein are directed to preventing and/or mitigating musculoskeletal disorders using a sensor-enabled system that dynamically alters an external geometry of body-wearable actuatable components. Based on sensor measurements, interventions of relatively short duration (“micro-interventions”) may be provided to control the alignment of the relevant body system of the wearer during motion. In this way, the alignment may be returned to a correct alignment and/or gradually altered to strengthen the body system and/or use the biomechanically correct muscle groups. While the discussion is primarily focused on implementations of a foot-worn apparatus that is dynamically adjusted within gait cycles of the wearer, the person of ordinary skill will recognize that the techniques described herein may be applied to other types of body-worn apparatus to affect other body systems during motion.

One means to affect gait is to add and/or remove support for the arch of a wearer's foot. Shoes have traditionally provided a static support for the arch; however, the arch is a dynamic portion of the foot during gait. There are complex interactions between the metatarsals, cuneiforms, cuboid, plantar fascia, and other musculoskeletal structures throughout stance and swing phases. For example, during a mid-stance phase the plantar aponeurosis and flexor digitorum longus tendons (the plantar fascia) are somewhat relaxed, but at a late-stance phase the plantar fascia stretches, storing energy in its high tension. A static arch can counteract this stretching, applying a transverse load to the tendons and potentially aggravating inflammation or other issues. A dynamic arch support mechanism can potentially provide support when needed, and stop applying support to the foot when necessary to better accommodate the natural behavior of the body. Providing dynamic arch support can also help align the foot during stance phases such that the ground reaction forces in the leg are directed toward the proximal joints to reduce loading that contributes to or exacerbates knee osteoarthritis. Dynamically aligning the foot can reduce the loading at the knee and ultimately encourage improved musculoskeletal alignment further up the kinematic chain of the body. In some cases, the dynamic arch may be able to alter a timing of the gait, e.g., to more closely replicate barefoot walking.

Measurements of the motion of the wearer's body may be acquired to determine how the micro-interventions at the foot or knee may affect the body. In a controlled (e.g., laboratory) setting, optical tracking systems such as the Vicon system may be used. Reflective markers may be used with infrared tracking cameras to measure sub-millimeter accuracy near 100 hertz (Hz). These are considered the highest accuracy, and a standard from which to estimate pose, position, and motion. Additionally, force measurement plates built into the floor (or treadmill) with six degrees-of-freedom measurements can calculate ground reaction force vectors. The combination of the optical tracking and ground reaction forces can be combined to estimate the kinematics and kinetics of dynamic motion.

After deployment of the sensor-enabled system, the accuracy of measurements may be much less than that of the controlled setting. Measurement devices used may include inertial measurement units (IMU) (measuring acceleration and rotation to estimate angle in space) and/or goniometers (measuring joint angle). The IMU typically senses some combination of acceleration (in up to six axes), gyroscopes (rotational velocity), magnetometer (heading), and barometers (altitude). Today, model-based sensor fusion enables accuracy of orientation measurements (angles) on the order of 1-2 degrees at best, but a spatial position of the limb segment measurements can be almost impossible to estimate accurately over time and with movement. The position is calculated from a double integration of acceleration, thus the measurement is prone to inaccuracy and drift over time. The constants of integration from each time step are assumed, which contributes to inaccuracy. The longer time period over which data is collected, the further the error tends to grow. If an IMU is resting on the ground and is initially calibrated it may maintain an accuracy of 2-3 millimeters (mm) for a minute or more, but without re-calibration this value may drift substantially over time. Further, IMUs tend to be more accurate when kept away from other metal objects. This is due to the magnetometer which tracks the magnetic field of the Earth, but can be diverted by local magnetic fields such as stairways, fire hydrants, or any other nearby metal objects. Goniometers provide angle measurement by directly measuring the angular of displacement of a joint. Compact implementations of the goniometers generally can measure motion in 2 degrees of freedom, but may not perform as well at measuring small motions such as the rotation about the long axis of the knee or the anterior draw (fore-aft motion of the knee). Although other types of sensors may be used in conjunction with the goniometers to measure the small motion, such sensors may be prohibitively expensive and/or bulky. The goniometers also require crossing a joint, and are not able to measure the orientation of the limb segment center of mass, for example, but are instead better at measuring readily-defined and 1 or 2 degrees of freedom-dominated motions, again such as the knee. Additionally, sensors may be placed on the bottom of the foot to determine a pressure distribution over time, a location, and/or a duration during a wearer's gait cycle. An example of such a sensor is an insole fitted with many resistive or capacitive “switches” overlapping the surface of the foot. As the foot applies pressure to each location in the insole, the “switch” measures the change in resistance or capacitance at that location. By taking measurements from all positions simultaneously, and comparing those measurements over time, an accurate estimation of which portion of the foot conveying load to the walking surface over time. While this technique may be susceptible to extraneous “noise,” such as measurements which change due to temperature or moisture within the insole, and while this technique may not be very accurate in providing absolute measurements for a particular subject, this technique does provide good relative measurements. Relative measurements show with a high degree of accuracy exactly when loads are conveyed by locations on the bottom of the foot, which enables detection of a gait phase during deployment with acceptable accuracy.

To increase a measurable accuracy of the micro-interventions described, improved accuracy of measurement for a mobile sensor suite may be needed. In some embodiments, body-wearable sensors may be arranged at several positions so that they can provide maximal benefit in order to measure the kinematics and kinetics of a person. In order to apply micro-interventions to affect the kinematic chain it is important to represent the motion of the wearer as accurately as possible over time. This generally requires collecting data at a high rate and a high accuracy at multiple locations across the body.

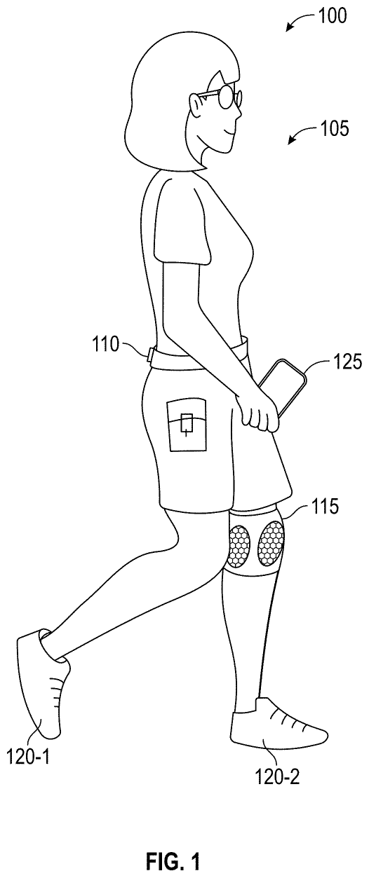

FIG. 1 illustrates an exemplary environment 100 for dynamically altering an external geometry of body-wearable actuatable components during gait cycles, according to one or more embodiments.

In the environment 100, a wearer 105 wears a plurality of sensor devices during walking. The plurality of sensor devices include a waist-worn sensor device 110, a knee-worn sensor device 115, and foot-worn sensor devices 120-1, 120-2 that are communicatively coupled with a mobile computing device 125 carried by the wearer 105. As shown, the waist-worn sensor device 110 is implemented as a waist clip that attaches to clothing of the wearer 105, the knee-worn sensor device 115 is implemented as a flexible sleeve, and the foot-worn sensor devices 120-1, 120-2 are implemented as insoles inserted into shoes of the wearer 105. Other implementations of the plurality of sensor devices are also contemplated, e.g., integrated into clothing of the wearer 105. For example, the foot-worn sensor devices 120-1, 120-2 may be integrated into the midsoles of the shoes of the wearer 105.

The mobile computing device 125 may be implemented in any suitable form, e.g., a hand-held or wearable computing device. Some non-limiting examples of the mobile computing device 125 include a smartphone, a tablet computer, and smartglasses. In some embodiments, the mobile computing device 125 wirelessly receives sensor signals from the plurality of sensor devices, detects a phase of the gait cycle from the sensor signals, and wirelessly transmits control signals to the plurality of sensor devices based on the detected phase to alter the external geometry of the one or more body-worn actuatable components. In this way, the control signals configure the plurality of sensor devices to provide micro-interventions within the gait cycles of the wearer 105. In some embodiments, the plurality of sensor devices provide different micro-interventions (e.g., the external geometry is altered differently) for different phases of the gait cycles.

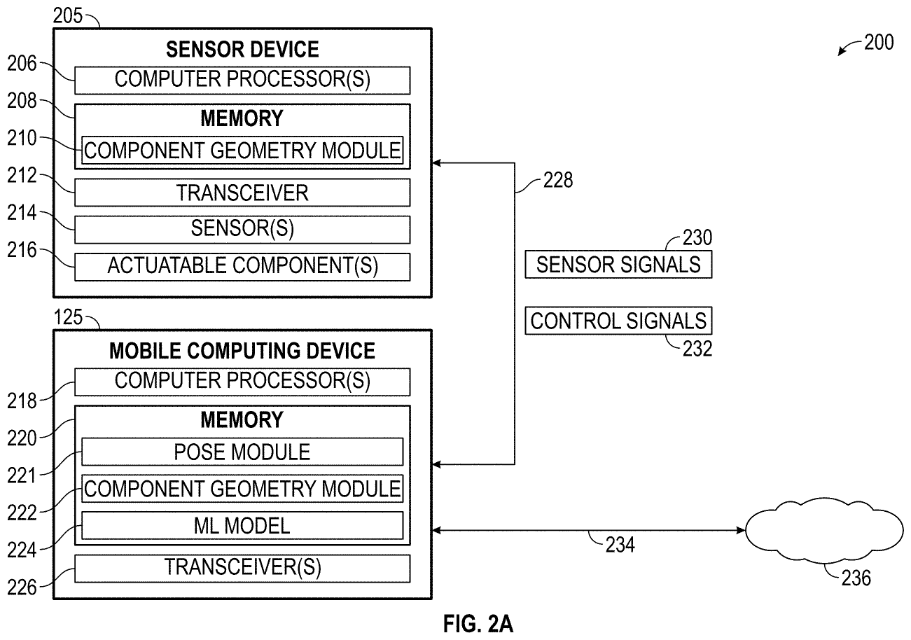

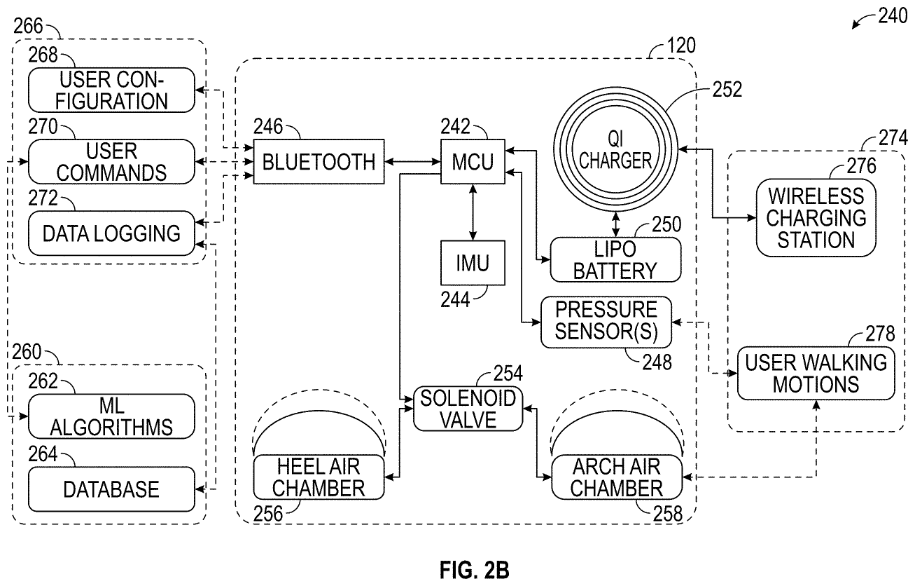

FIG. 2A is a system 200 of dynamically altering an external geometry of body-wearable actuatable components during gait cycles, according to one or more embodiments. The features illustrated in FIG. 2A may be used in conjunction with other embodiments.

The system 200 comprises a sensor device 205 communicatively coupled with the mobile computing device 125 via a communicative link 228. The sensor device 205 represents one of the waist-worn sensor device 110, the knee-worn sensor device 115, and the foot-worn sensor devices 120-1, 120-2 of FIG. 1. Although not shown, the mobile computing device 125 may be communicatively coupled with one or more other sensor devices.

The sensor device 205 comprises one or more computer processors 206, a memory 208, a transceiver 212, one or more sensors 214, and one or more actuatable components 216. The one or more computer processors 206 may be implemented in any suitable form, such as a general-purpose microprocessor, a controller, an application-specific integrated circuit (ASIC), and so forth. The memory 208 may include a variety of computer-readable media selected for their size, relative performance, or other capabilities: volatile and/or non-volatile media, removable and/or non-removable media, etc.

The memory 208 may include one or more modules for performing various functions described herein. In one embodiment, each module includes program code that is executable by the one or more computer processors 206. In another embodiment, each module is partially or fully implemented in hardware (i.e., circuitry) or firmware of the sensor device 205 (e.g., as circuitry within the one or more computer processors 206). However, other embodiments of the system 200 may include modules that are partially or fully implemented in other hardware or firmware, such as hardware or firmware included in one or more other computing devices connected with the network, and so forth. Stated another way, the overall functionality of the one or more modules may be distributed among other devices of the system 200.

The transceiver 212 comprises circuitry that is communicatively coupled with the one or more computer processors 206 and configured to transmit and receive electrical or optical signals via the communicative link 228. The communicative link 228 may have any suitable implementation, such as copper transmission cable(s), optical transmission fiber(s), wireless transmission, router(s), firewall(s), switch(es), gateway computer(s), and/or edge server(s). In some embodiments, the transceiver 212 comprises a wireless transceiver.

The one or more sensors 214 may comprise sensors of any suitable type(s). In some embodiments, the one or more sensors 214 comprise pressure sensors. The one or more actuatable components 216 may, responsive to receiving control signals, actuate using any suitable techniques to dynamically alter an external geometry during gait cycles. In some embodiments, the one or more actuatable components 216 comprise one or more inflatable bladders and/or one or more rotational actuators. In some embodiments, the one or more actuatable components 216 comprise a plurality of sections (e.g., a segmented bladder) that may be individually controlled.

As shown, the memory 208 comprises a component geometry module 210 that determines values for dynamically altering the external geometry of the one or more actuatable components 216. In some embodiments, the mobile computing device 125 comprises a component geometry module 222 that is configured similarly to the component geometry module 210. In some embodiments, sensor signals 230 from the one or more sensors 214 are applied to a model to determine the values. In some embodiments, the model comprises a machine learning model 224, which may be trained using sensor data from the wearer and/or one or more other wearers. The values may be represented in any suitable forms, such as pressure set points, dimensions or sizes of the one or more actuatable components 216 (e.g., height values), and so forth. In some embodiments, the sensor device 205 transmits sensor signal(s) 230 to the mobile computing device 125 via the communicative link 228, and receives control signals 232 from the mobile computing device 125 via the communicative link 228. In some alternate implementations, the component geometry module 210 generates the control signals 232 for controlling the one or more actuatable components 216 according to the values.

The mobile computing device 125 comprises one or more computer processors 218, a memory 220, and one or more transceivers 226. In some embodiments, the one or more computer processors 218 may be configured similarly to the one or more computer processors 206 discussed above, and the memory 220 may be configured similarly to the memory 208 discussed above. In some embodiments, the performance of the one or more computer processors 206 and the memory 208 is limited to conserve battery life of the sensor device 205, and the one or more computer processors 218 and/or the memory 220 offers relatively greater performance. In this way, the computing workload(s) may be offloaded from the sensor device 205.

In some embodiments, the memory 220 comprises a pose module 221 that is configured to determine and/or predict poses for one or more body parts of the wearer using the sensor signals 230. As defined herein, a “pose” represents a disposition of one or more body parts of the wearer in two-dimensional or three-dimensional space. In some embodiments, each pose includes position information and orientation information for the one or more body parts.

In one non-limiting example, the pose module 221 determines and/or predicts poses for a leg and/or a foot of the wearer. In some embodiments, the one or more sensors 214 comprise one or more sensors arranged above the foot (e.g., at a proximal position along the leg, on the torso of the wearer, and so forth), and the pose module 221 determines and/or predicts poses of the leg and/or the foot using sensor signals 230 received from the one or more sensors arranged above the foot. In some embodiments, the pose module 221 uses sensor signals 230 received from the one or more sensors arranged above the foot and from one or more foot-worn sensors.

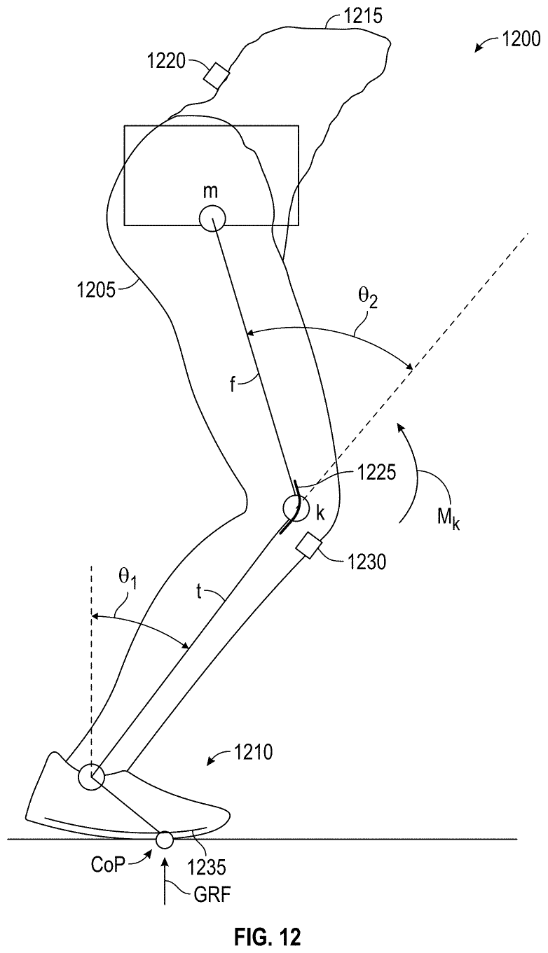

In some embodiments, the pose module 221 applies the sensor signals 230 to a biomechanical model representing the wearer to determine and/or predict the poses. One exemplary technique for determining a pose of a leg and foot of a wearer is illustrated in FIG. 12. A leg 1205, a foot 1210, and a portion of a torso 1215 of the wearer are depicted in diagram 1200.

In some embodiments, the pose module 221 may estimate one or more internal forces of the lower-extremity musculoskeletal system, and in some cases may combine those force estimate(s) into a single numerical value that estimates knee health of the wearer in real-time. The pose module 221 receives sensor signals from one or more body-worn sensors, which in the diagram 1200 include an IMU 1220 worn on the torso 1215 (one example of the waist-worn sensor device 110 of FIG. 1), a bend sensor 1225 and an IMU 1230 (examples of the knee-worn sensor devices 115 of FIG. 1), and a ground sensor 1235 (one example of the foot-worn sensor device 120 of FIG. 1). Other compositions of the one or more body-worn sensors are also contemplated.

In some embodiments, the one or more body-worn sensors may sample each of the variables required for a forward kinematic analysis of the lower-extremities that can be used to calculate knee health. Calculating the resultant forces from the ground and center of mass enable an external moment estimate at the knee that can be used to estimate the additional internal loads due to muscle-tendon forces required to support the body.

Combination of the sensor signals and pre-measured or estimated biomechanical data of the wearer may be used to calculate the reaction forces and moments acting at the knee. In some embodiments, the ground sensor 1235 may identify when the foot 1210 is stationary on the ground during a stride. In some embodiments, the ground sensor 1235 may also measure an equivalent ground reaction force (F_{g}), and a location of the resultant force is the center of pressure (CoP), which combine to provide a ground reaction force vector. The IMU 1230 provides a measurement of a tibia pitch angle (θ₁) which may be used to determine an angle of the ankle. In one non-limiting example, the tibia pitch angle θ₁ equals the angle of the ankle. The ankle angle, combined with a tibia length (t), describes the position of the knee in space. The bend sensor 1225 measures a knee angle (θ₂) and may be combined with a femur length (f) to provide an estimate of the position of the pelvis and a center of mass. The IMU 1220 located at the pelvis can also be used to estimate an orientation of the torso 1215, which can also identify and/or predict gait pathologies or changes in terrain.

In some alternate implementations, another IMU may be arranged at the foot 1210 to provide two additional measurements: a ground angle measurement during a mid-stance phase (stationary foot) would inform ground conditions, and a position of the IMU may be tracked in space to identify the level of pronation in each step or variations in terrain such as stairs or slopes.

Generally, more sophisticated inverse kinematic analysis is not required as multi-joint path-planning trajectories are not calculated. Instead, external kinetics and kinematics are measured to estimate internal kinetics at a single joint (e.g., the knee). High precision is not required as multiple joints are not being simultaneously controlled. In some embodiments, the micro-interventions occurring at the foot are applied against the body of the wearer which is a highly dynamic, self-adjusting system. In this way, the micro-interventions effectively urge the body toward a target pose, which augments the ability of the body of the wearer to adjust on its own.

In some embodiments, the musculoskeletal moment to support the weight of the wearer's body due to quadriceps muscle-tendon complex may be calculated by summing moments about the center of rotation of the knee:

ΣMₖ: 0=−F_{g}[t·cos(θ₁)−COP]+f·m·cos(θ₁+θ₂)+M

M=F_{g}·(t·cos(t·cos(θ₁)−COP)−f·m·cos(θ₁+θ₂)

The patellar tendon resultant tension force may be calculated as:

$$T = \frac{M}{p}$$

The internal force on the condyle may be calculated as:

Fᵢₙₜₑᵣₙₐₗ=F_{G}+T

Estimating the contact area, A_{c}, of the condyle as a function of joint angle may be used to estimate stress, σₖ, at the cartilage interface, which in some cases may serve as a proxy for knee pain:

$${σ}_{k} = \frac{{F}_{i n t e r n a l}}{{A}_{c} ( {θ}_{2} )}$$

For the above calculations, several assumptions may be made: the instantaneous center of rotation of the knee may be assumed statically located instead of tracking the real progression. The patellar distance (p) and the condyle contact area may be assumed as constants, but in alternate implementations may be updated based on angle and/or force dependent data. The moment at the knee supporting the weight of the wearer's body may be assumed to be contributed entirely from the quadriceps tendon, omitting added forces that may exist to provide knee joint stiffness but net zero external moment force. Further, static equilibrium may be assumed for each calculation step, despite dynamic motion clearly being in effect.

In some embodiments, the numerical value that estimates knee health of the wearer may be a knee index that characterizes articular cartilage loading at the tibio-femoral interface, represented by the resultant knee adduction moments (KAM) and knee flexion moments (KFM). The cumulative damage caused by these two loading conditions contribute to progression of knee osteoarthritis and impulse external moments (time integral over mid-stance phase) may be better predictors than peak external moments for disease progression. Generally, KAM may be a better predictor but more difficult to measure in vivo (due to a smaller range of motions), and the KFM may be an accepted predictor. A reduction in cumulative damage may delay the onset of knee osteoarthritis progression.

Using an implementation of the foot-wearable apparatus described herein, the micro-interventions provided by the foot-wearable apparatus may provide more than a 20% reduction in total damage per stride (when compared to no intervention), calculated using an integral of a total knee moment as calculated by the knee index. Further, gait behavior modifications that intervene to roll the ankle medially or laterally to adjust medial-lateral loading at the tibio-femoral interface may further improve KAM loading. The dynamic control of the external geometry of the foot-wearable apparatus may provide this medial-lateral intervention specific to each wearer's biomechanical needs.

Returning to FIG. 2A, in one example the pose module 221 determines and/or predicts a pose (e.g., for the leg and/or foot of the wearer) based on the one or more sensor signals 230, and compares the pose with a target (or desired) pose. In some cases, the target pose corresponds to a phase or portion of a gait cycle, or of another predefined movement. Based on a determined difference between the pose and the target pose, the component geometry module 222 alters the external geometry of the one or more actuatable components 216 to adjust the leg of the wearer toward the target pose during the motion. In some embodiments, the component geometry module 222 alters the external geometry of the one or more actuatable components 216 according to multiple settings (corresponding to multiple target poses) within the gait cycle or predefined movement.

In some embodiments, the target pose(s) may be stored in a memory that is worn or carried by the wearer, such as the memory 208. In some embodiments, the target pose(s) may be determined for the wearer in a clinical setting, e.g., by a healthcare professional such as a doctor or therapist, and stored in the memory 208. In some embodiments, the target pose(s) may be determined and/or altered dynamically during wear. In some embodiments, the target pose(s) may be determined responsive to feedback from the wearer. For example, during wear the wearer may provide inputs to the mobile computing device 125 responsive to pain or discomfort. In some embodiments, the wearer may specify by the inputs the particular alterations to be made to the one or more actuatable components 216. In one example, the target poses may be displayed on the mobile computing device 125 and the wearer may graphically adjust the target poses (e.g., rotating a graphical representation of the leg to adjust an angle of the target poses). In another example, the wearer may specify the adjustment to be made (e.g., increase inflation by 10%). In another example, the wearer may indicate a location of pain or discomfort, and the pose module 221 determines adjustment(s) to target pose(s) that are executed using the component geometry module 222.

In some embodiments, the sensor device 205 may be operated while maintaining an active communicative connection to the mobile computing device 125 and/or a network 236. In other embodiments, the target pose(s) and/or the corresponding settings for the one or more actuatable components 216 may be downloaded and/or stored in the memory 208 such that the sensor device 205 may be operated without requiring an active communicative connection to the mobile computing device 125 and/or the network 236. In this case, the sensor device 205 may communicate stored data, updated settings, and so forth once the active communicative connection to the mobile computing device 125 and/or the network 236 is available.

In one simplified, non-limiting example of operation of the pose module 221, the pose module 221 determines an overpronation of the wearer's foot in a mid-stance phase of a gait cycle. The overpronation tends to cause the wearer's ankle to roll inward during the mid-stance phase. Based on a target pose stored in the memory 208 for the mid-stance phase (e.g., a neutral position), the pose module 221 may communicate with the component geometry module 222 to cause a longitudinal arch support to inflate or to otherwise change its shape to counter the tendency to overpronate. By altering the external geometry of the longitudinal arch support, the system 200 may urge the wearer's foot toward the target pose (e.g., neutral position) during the mid-stance phase. As discussed in greater detail herein, the external geometry of the one or more actuatable components 216 may be dynamically altered for different phases of the gait cycle and/or other predefined movements. In some embodiments, the memory 208 of the sensor device 205 and/or the memory 220 of the mobile computing device 125 stores one or more target poses that are accessed by the pose module 221. In some embodiments, some or all of the functionality of the pose module 221 may be implemented in the memory 208 of the sensor device 205.

In some embodiments, the one or more transceivers 226 may be configured similarly to the transceiver 212 discussed above. In some embodiments, a first transceiver of the one or more transceivers 226 is communicatively coupled with the transceiver 212 via the communicative link 228, and a second transceiver of the one or more transceivers 226 is communicatively coupled with a network 236 via a communicative link 234. In some embodiments, the communicative link 234 is configured similarly to the communicative link 228 discussed above.

The network 236 represents one or more networks of any suitable types, such as the Internet, a local area network (LAN), a wide area network (WAN), and/or a wireless network. In some alternate embodiments, the functionality of the component geometry module 210, the component geometry module 222, and/or the ML model 224 is implemented in a computing device that is accessible through the network 236. Further, in some embodiments, the transceiver 212 of the sensor device 205 communicates directly with the network 236, such that the mobile computing device 125 may be omitted.

FIG. 2B is a system 240 of dynamically altering an external geometry of foot-wearable actuatable components during gait cycles, according to one or more embodiments. The features illustrated in FIG. 2B may be used in conjunction with other embodiments. For example, the system 240 represents one possible implementation of the system 200 of FIG. 2A.

A foot-worn sensor device 120 (representing one of the foot-worn sensor devices 120-1, 120-2) comprises a microcontroller unit (MCU) 242 (representing one example of the one or more computer processors 206). The foot-worn sensor device 120 further comprises an IMU 244 (representing one example of the one or more sensors 214), a Bluetooth transceiver 246 (representing one example of the transceiver 212), and two (2) pressure sensors 248 (representing another example of the one or more sensors 214) that are each communicatively coupled with the MCU 242. A lithium polymer (LiPo) battery 250 provides electrical power to the MCU 242, although other types of batteries are also contemplated. To charge the LiPo battery 250, user interactions 274 with the foot-worn sensor device 120 may include wirelessly coupling a Qi charger 252 of the foot-worn sensor device 120 to a wireless charging station 276. Alternate implementations of the foot-worn sensor device 120 may use different types of charging, such as wired charging.

The user interactions 274 with the foot-worn sensor device 120 may further include performing user walking motions 278, causing the pressure sensors 248 to transmit sensor signals to the MCU 242. The MCU 242 communicates, via the Bluetooth transceiver 246, with an application 266 on a mobile computing device. In some embodiments, the application 266 receives user configuration information 268, user commands 270, and data logging information 272 from the MCU 242. In some embodiments, the user configuration information 268 is received at the application 266 via one or more user inputs. The application 266 may communicate the data logging information 272 (e.g., sensor data acquired from the IMU 244 and/or the pressure sensors 248) to a database 264 of a cloud-based server 260. The database 264 may store information associated with the wearer, as well as one or more other wearers. One or more ML and/or artificial intelligence (AI) algorithms 262 of the cloud-based server 260 may generate the user commands 270 based on the database 264, and may communicate the user commands 270 to the application 266 of may download behaviors to the device for use during real-time interactions. The application 266 may adapt the user commands 270 based on the user configuration information 268, and may communicate the user commands 270 to the MCU 242. Other types of user interactions 274 are also contemplated, such as user jumping, running, and/or hopping motions.

In some embodiments, the foot-worn sensor device 120 further comprises a heel air chamber 256 and an arch air chamber 258 (representing examples of the one or more actuatable components 216). During gait cycles, the user walking motions 278 may further cause pressure changes in the heel air chamber 256 and/or in the arch air chamber 258. Using the user commands 270, the MCU 242 generates control signals to operate a solenoid valve 254 to transfer fluid between the heel air chamber 256 and the arch air chamber 258. For example, during a heel strike phase of a gait cycle, the heel of the user strikes the ground surface causing the pressure in the heel air chamber 256 to increase substantially, and the control signals may control the solenoid valve 254 to transfer fluid from the higher pressure heel air chamber 256 to the lower pressure arch air chamber 258, allowing the external geometry of the arch air chamber 258 to be altered prior to a mid-stance phase of the gait cycle where the ball of the foot contacts the ground surface. During a push-off phase of the gait cycle, the heel of the user is raised off the ground and the arch air chamber 258 is at a higher pressure than the heel air chamber 256. The control signals may control the solenoid valve 254 to transfer fluid from the higher pressure arch air chamber 258 to the lower pressure heel air chamber 256, allowing the external geometry of the heel air chamber 256 to be altered prior to a heel strike phase of a next gait cycle. Although the solenoid valve 254 is described as providing two-way fluid flow, alternate embodiments may use multiple one-way valves to achieve the fluid flow between the heel air chamber 256 and the arch air chamber 258.

In some implementations, the MCU 242 may further generate control signals to operate an active fluid source (e.g., a pump or a pressurized air supply) to supply (additional) pressurized air to the heel air chamber 256 and/or the arch air chamber 258 in conjunction with operation of the solenoid valve 254, e.g., to control an external geometry of the heel air chamber 256 and/or the arch air chamber 258. For example, the MCU 242 may determine that, in a mid-stance phase of a subsequent gait cycle, the arch air chamber 258 will be inflated to a greater inflation state than in the mid-stance phase of a previous gait cycle. If an insufficient pressure can be generated in the arch air chamber 258 responsive to the heel strike phase (and operation of the solenoid valve 254), the active gas source may be operated to inject a desired amount of air into the arch air chamber 258 to achieve the greater inflation state. Similarly, the MCU 242 may further generate control signals to operate a valve to vent air and reduce pressure in the bladder system.

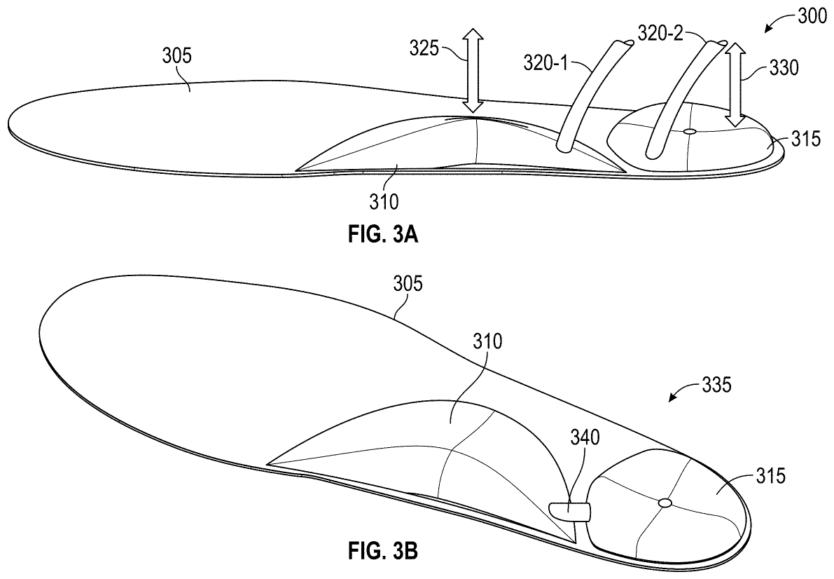

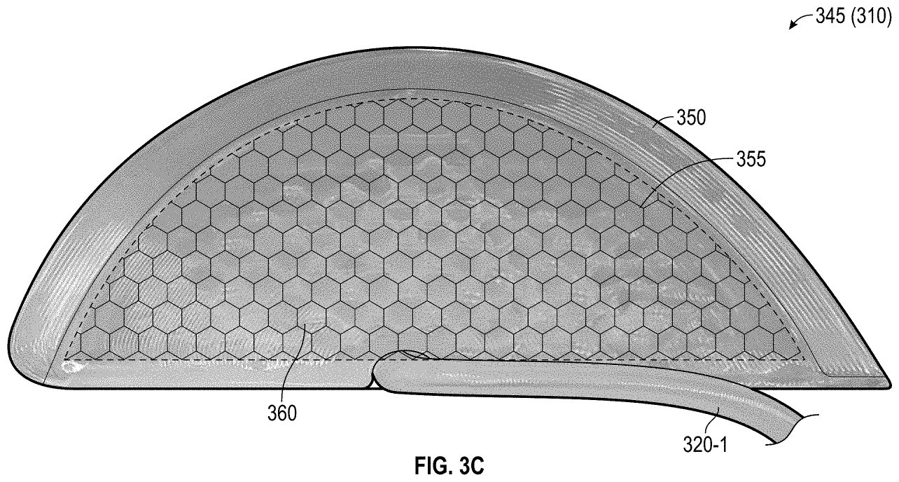

FIG. 3A illustrates a foot-wearable apparatus 300 with independent inflatable bladders, according to one or more embodiments. The features illustrated in FIG. 3A may be used in conjunction with other embodiments. For example, the foot-wearable apparatus 300 may represent one possible implementation of the foot-worn sensor device 120 discussed in FIGS. 1, 2A, and 2B.

The foot-wearable apparatus 300 comprises an insole 305 coupled with a longitudinal arch support 310 and a heel support 315. Each of the longitudinal arch support 310 and the heel support 315 comprises an inflatable bladder that may be actuated during gait cycles to dynamically alter the external geometry of the foot-wearable apparatus 300. In some embodiments, the inflatable bladders are inflated using environmental air, although other suitable fluids are also contemplated (whether gases, liquids, or combinations thereof). Further, alternate implementations of the foot-wearable apparatus 300 may include different types of actuating materials, such as indium gallium soft actuators having suspended air bubbles that heat when expanded.

The insole 305, the longitudinal arch support 310, and the heel support 315 may be formed of any suitable material(s). In some embodiments, the insole 305, the longitudinal arch support 310, and the heel support 315 are integrally formed of a rubber material. In other embodiments, the insole 305, the longitudinal arch support 310, and the heel support 315 may be separately formed and attached to each other, and/or may be formed of other material(s). Further, alternate implementations of the foot-wearable apparatus 300 may be integrated into an article of footwear. In one example, the longitudinal arch support 310 and/or the heel support 315 may be integrated into a midsole of a shoe and the actuation may deform a flexible insole to present a desired external geometry to the foot of the wearer. In another example, the longitudinal arch support 310 and/or the heel support 315 may be integrated into a sock worn by the wearer.

The foot-wearable apparatus 300 further comprises a fluid port 320-1 in fluid communication with the inflatable bladder of the longitudinal arch support 310, and a fluid port 320-2 in fluid communication with the inflatable bladder of the heel support 315. In some cases, the fluid ports 320-1, 320-2 are implemented as tubes connected with the respective one of the longitudinal arch support 310 and the heel support 315. In some embodiments, each of the fluid ports 320-1, 320-2 is also in fluid communication with a fluid source (e.g., a manual or electric pump) or activation energy source configured to provide fluid to inflate the respective inflatable bladder to a desired inflation state. Inflation or deflation of the longitudinal arch support 310 may cause actuation along an axis 325, and inflation or deflation of the heel support 315 may cause actuation along an axis 330. In alternate implementations, the foot-wearable apparatus 300 may be actuated using an electrorheological fluid, a shape-memory metal, an indium gallium soft actuator, and so forth.

Although the longitudinal arch support 310 and the heel support 315 are described as having a single inflatable bladder, alternate implementations of the foot-wearable apparatus 300 may have multiple inflatable bladders in the longitudinal arch support 310 and/or the heel support 315. In this way, the shape of the longitudinal arch support 310 and/or the heel support 315 may be controlled, in addition to a same inflation of the multiple inflatable bladders.

Alternate implementations of the foot-wearable apparatus 300 may include different arrangements of actuatable components. In one example, the longitudinal arch support 310 or the heel support 315 may be omitted. In another example, the foot-wearable apparatus 300 may include a transverse arch support having inflatable bladder(s). Further, although the longitudinal arch support 310 or the heel support 315 are arranged at discrete locations along the insole 305, alternate implementations of the foot-wearable apparatus 300 may include multiple inflatable bladders that are continuously distributed along one or more regions of the insole 305.

FIG. 3B illustrates a foot-wearable apparatus 335 with inflatable bladders in fluid communication, according to one or more embodiments. The features illustrated in FIG. 3B may be used in conjunction with other embodiments. For example, the foot-wearable apparatus 335 may represent one possible implementation of the foot-wearable apparatus 120 discussed in FIGS. 1, 2A, and 2B.

In the foot-wearable apparatus 335, a fluid port 340 (e.g., a tube) provides fluid communication between the longitudinal arch support 310 and the heel support 315. Although not shown, in some embodiments, a valve may be in communication with the fluid port 340 to selectively transfer gas between the longitudinal arch support 310 and the heel support 315. Although not shown, the longitudinal arch support 310 and/or the heel support 315 may include one or more additional fluid ports to intake or vent fluid into the bladder system.

FIG. 3C illustrates a longitudinal arch support 345 having an inflatable bladder, according to one or more embodiments. The features illustrated in FIG. 3C may be used in conjunction with other embodiments. For example, the longitudinal arch support 345 may represent one possible implementation of the longitudinal arch support 310 discussed in FIGS. 3A and 3B.

The longitudinal arch support 345 comprises a sealed exterior 350 defining an interior volume 355 in fluid communication with the fluid port 320-1. The longitudinal arch support 345 further comprises a cellular structure 360 within the interior volume 355, which may be formed of a flexible material. The longitudinal arch support 345 may include other types of structural elements, whether disposed within the interior volume 355 or external to it. The structural elements may assist to preferentially inflate and/or deflate the longitudinal arch support 345, to prevent shearing of the longitudinal arch support 345. For example, the longitudinal arch support 345 may include walls of differing strengths, a webbing preventing deformation of the longitudinal arch support 345 in one or more directions, and so forth.

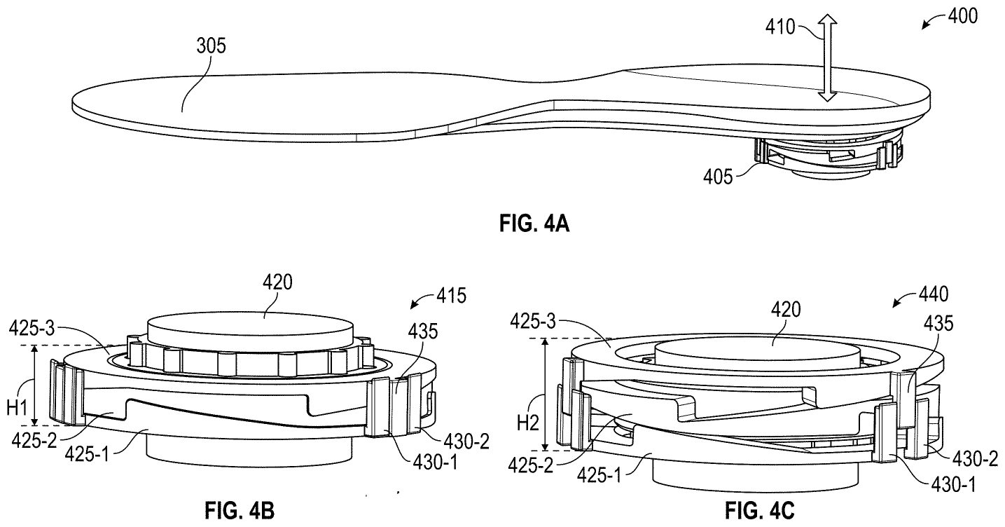

FIG. 4A illustrates a foot-wearable apparatus 400 having a heel support with a rotational actuator 405, according to one or more embodiments. The features illustrated in FIG. 4A may be used in conjunction with other embodiments.

As shown, the rotational actuator 405 is coupled to a bottom surface of the insole 305. The rotational actuator 405 provides an axial displacement along an axis 410. Further, although the rotational actuator 405 is shown as a sole actuatable device of the foot-wearable apparatus 400, alternate implementations of the foot-wearable apparatus 400 may include one or more other actuatable devices, such as a longitudinal arch support (e.g., including an inflatable bladder).

FIG. 4B illustrates the rotational actuator 405 in a retracted configuration, and FIG. 4C illustrates the rotational actuator 405 in an extended configuration, according to one or more embodiments. In diagram 415, the rotational actuator 405 comprises a central rotor 420 and a plurality of stacked rings 425-1, 425-2, 425-3 arranged around the central rotor 420. Although not shown, the central rotor 420 may be rotated using a motor. The stacked ring 425-2 is configured to rotate with the central rotor 420, while the stacked rings 425-1, 425-3 are rotationally constrained. In an alternate implementation, the stacked ring 425-2 may be replaced by a stacked disk and may be integrally formed with the central rotor 420.

The stacked rings 425-1, 425-2, 425-3 are slidingly coupled with each other, and the faces of the stacked rings 425-1, 425-2, 425-3 are contoured such that rotation of the stacked ring 425-2 causes an axial displacement between the stacked rings 425-1, 425-3. The stacked ring 425-1 is coupled with splines 430-1, 430-2 that are spaced apart from each other along the outer circumference of the stacked rings 425-1, 425-2, 425-3. The stacked ring 425-3 is coupled with a spline 435 arranged between the splines 430-1, 430-2. The splines 430-1, 430-2 limit the movement of the spline 435, causing the stacked rings 425-1, 425-3 to be rotationally constrained.

In the retracted configuration of FIG. 4B, a bottom face of the stacked ring 425-1 and a top face of the stacked ring 425-3 are axially separated by a height h1. In the extended configuration of FIG. 4C, the bottom face of the stacked ring 425-1 and a top face of the stacked ring 425-3 are axially separated by a height h2 that is greater than the height h1. In this way, a corresponding heel portion of the insole 305 contacting the stacked ring 425-3 may be raised or lowered within gait cycles. Although the rotational actuator 405 is shown as a single stack of stacked rings 425-1, 425-2, 425-3, alternate implementations of the foot-wearable apparatus 400 may include multiple stacks of stacked rings.

Further, although not shown, the top face of the stacked ring 425-3 may be dynamically oriented as part of altering the external geometry of the actuatable component(s). For example, the top face of the stacked ring 425-3 may be flat relative to the axis 410 and the entire rotational actuator 405 may be dynamically reoriented, and/or the top face of the stacked ring 425-3 may be slanted relative to the axis 410.

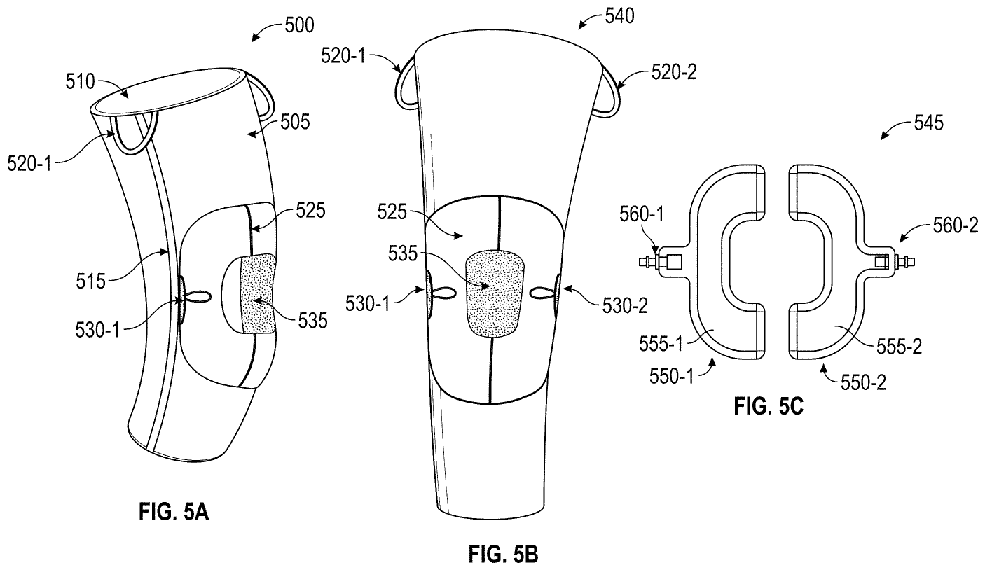

FIG. 5A is a diagram 500 illustrating a perspective view, and FIG. 5B is a diagram 540 illustrating a front view, of a knee-wearable apparatus according to one or more embodiments. FIG. 5C is a diagram 545 illustrating two inflatable bladders for the knee-wearable apparatus. The features illustrated in FIGS. 5A-5C may be used in conjunction with other embodiments. For example, the knee-wearable apparatus represents one example implementation of the knee-worn sensor device 115 of FIG. 1.

The knee-wearable apparatus comprises a sleeve 505 defining an upper opening 510 at the superior end, and through which a wearer's leg may be inserted when donning the knee-wearable apparatus. The sleeve 505 further defines a seam 515 extending along a length of the knee-wearable apparatus on a lateral side of the knee-wearable apparatus. The sleeve 505 and the seam 515 may be formed of any suitable material(s). In some embodiments, the sleeve 505 comprises a flexible fabric material and the seam 515 may be sewn, taped, or welded. Although not shown, the sleeve 505 may include one or more additional features that assist with secure and/or comfortable wear of the knee-wearable apparatus. For example, the sleeve 505 may include ribbed bands near superior and/or inferior ends of the knee-wearable apparatus, small shapes of material (e.g., silicone) disposed along an interior surface of the sleeve 505, and so forth.

Two handles 520-1, 520-2 are attached to the sleeve 505 near the superior end of the knee-wearable apparatus. The handles 520-1, 520-2 may be pulled by the wearer to assist with donning the knee-wearable apparatus. A pocket 525 is coupled to the sleeve 505 near a kneecap position on the anterior side of the knee-wearable apparatus. The pocket 525 may be attached or adhered to the sleeve 505, integrated into the sleeve 505, and so forth. In some embodiments, an opening 535 is defined in the pocket 525 that exposes the kneecap of the wearer. In other embodiments, the fabric of the sleeve 505 may extend over the kneecap.

The pocket 525 defines one or more interior volumes into which one or more inflatable bladders 550-1, 550-2 may be installed. In some embodiments, the inflatable bladders 550-1, 550-2 may be substantially permanently installed into the pocket 525 (e.g., inserted during manufacture of the knee-wearable apparatus) or removably installed (e.g., able to be removed or replaced by the wearer). The inflatable bladders 550-1, 550-2 comprise respective interior volumes 555-1, 555-2 that are in fluid communication with respective fluid ports 560-1, 560-2. The inflatable bladders 550-1, 550-2 may have any suitable dimensioning and/or arrangement. As shown, the inflatable bladders 550-1, 550-2 are C-shaped and oriented such that the inflatable bladders 550-1, 550-2 collectively substantially surround the kneecap position. Alternate implementations of the knee-wearable apparatus may have different shapes of the inflatable bladders 550-1, 550-2, different numbers of the inflatable bladders 550-1, 550-2, each the inflatable bladders 550-1, 550-2 may have a plurality of individually-inflatable chambers, and so forth.

In some embodiments, the fluid ports 560-1, 560-2 extend through lateral openings that are defined in the pocket 525. In some embodiments, the fluid ports 560-1, 560-2 comprise controllable valves enabling pressure control of the inflatable bladders 550-1, 550-2. In some embodiments, flexible tubing may connect the fluid ports 560-1, 560-2 to one or more external pumps. In alternate implementations, the fluid ports 560-1, 560-2 and/or the pump(s) may be integrated into the knee-wearable apparatus. Further, although the inflatable bladders 550-1, 550-2 are shown as being independently controlled, alternate implementations of the knee-wearable apparatus may have the inflatable bladders 550-1, 550-2 in fluid communication with each other. One or more valves may be operated to control fluid flow between the inflatable bladders 550-1, 550-2.

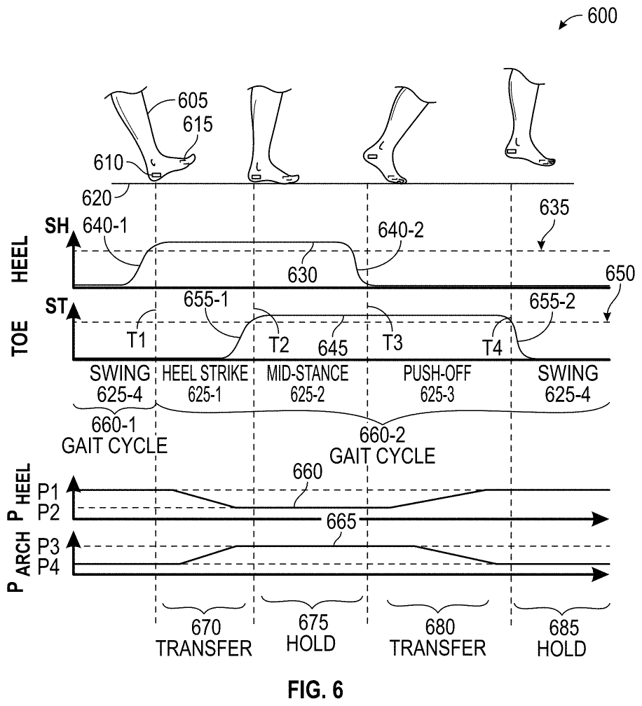

FIG. 6 is a diagram 600 illustrating sensor signals and pressure signals at different phases of gait cycles, according to one or more embodiments. The features illustrated in FIG. 6 may be used in conjunction with other embodiments. For example, the sensor signals may be acquired by the sensor device 205 of FIG. 2 and used to alter an external geometry of body-wearable actuatable components during gait cycles.

A top portion of the diagram 600 illustrates motion of a leg 605 in various poses relative to a ground surface 620. Although the poses depicted are representative of poses that may occur during gait cycles (e.g., during walking or running), poses occurring during other types of motion are also contemplated (e.g., hopping, jumping, dancing, skating, skiing, calisthenics, weight-lifting, and so forth). An aft sensor 610 (e.g., a “heel” sensor) generates a first sensor signal 630 and a forward sensor 615 (e.g., a “toe” sensor arranged near a ball of the foot) generates a second sensor signal 645. In some embodiments, the forward sensor 615 and/or the aft sensor 610 may be integrated into the foot-wearable apparatus 300, 335 of FIG. 3A, 3B or the foot-wearable apparatus 400 of FIG. 4A.

In some embodiments, the different phases occurring within the gait cycles 660-1, 660-2 may be identified using the sensor signals 630, 645. For example, during a swing phase 625-4 of the gait cycle 660-1, a first transition 640-1 of the sensor signal 630 occurs as the heel strikes the ground surface 620. At time t1, the sensor signal 630 exceeds a threshold value 635, which transitions from the swing phase 625-4 to a heel strike phase 625-1 of the gait cycle 660-2.

During the heel strike phase 625-1, a first transition 655-1 of the sensor signal 645 occurs as the ball of the foot strikes the ground surface 620. At time t2, the sensor signal 645 exceeds a threshold value 650, which transitions from the heel strike phase 625-1 to the mid-stance phase 625-2.

During the mid-stance phase 625-2, a second transition 640-2 of the sensor signal 630 occurs as the heel is lifted from the ground surface 620. At time t3, the sensor signal 630 is reduced below the threshold value 635, which transitions from the mid-stance phase 625-2 to the push-off phase 625-3.

During the push-off phase 625-3 (or “late-stance phase”), a second transition 655-2 of the sensor signal 645 occurs as the toe is lifted from the ground surface 620. At time t4, the sensor signal 645 is reduced below the threshold value 650, which transitions from the push-off phase 625-3 to the swing phase 625-4 of the gait cycle 660-2.

Although the first transitions 640-1, 655-1 and the second transitions 640-2, 655-2 are defined, respectively, relative to a single threshold value 635, 650, other implementations may have different threshold values for the first transition 640-1, 655-1 and the second transition 640-2, 655-2.

The micro-interventions may be provided by the body-wearable actuatable components based on detected and/or predicted poses. In some embodiments, one or more computer processors process the sensor signals 630, 645 to predict one or more poses. For example, the computer processor(s) may determine a timing (e.g., a periodicity and/or duration) of different phases of the gait cycles 660-1, 660-2 using the sensor signals 630, 645, and may predict various poses of the leg 605 and/or foot based on the timing.

In some embodiments, the computer processor(s) may detect a heel strike using the first transition 640-1 and may provide subsequent micro-interventions within the gait cycle 660-2 and/or subsequent gait cycles based on predicted poses. In other embodiments, the computer processor(s) may predict one or more poses associated with the heel strike phase 625-1 as an alternative to detecting the heel strike. In yet other embodiments, the computer processor(s) may predict the poses (e.g., using a ML model) without reference to predefined phases and/or timing.

In some embodiments, the micro-interventions may be provided by the body-wearable actuatable components to adjust the leg 605 toward a target pose during the motion. For example, the computer processor(s) may determine an alignment of the leg 605 based on the predicted pose(s), and may control the external geometry of the body-wearable actuatable components to provide the leg 605 with a “correct” (or other prescribed) alignment during the different phases of the gait cycles 660-1, 660-2. In another example, the external geometry of the body-wearable actuatable components is gradually altered (e.g., over multiple gait cycles) to strengthen the body system.

In some embodiments, the micro-interventions provided by the body-wearable actuatable components may differ based on the different phases of the gait cycles 660-1, 660-2. Generally, the micro-interventions provided by foot-worn actuatable devices may be most beneficial when provided during a maximum extension of the leg within the gait cycles 660-1, 660-2. For example, a failure to fully extend the leg or a misalignment at maximum extension may cause additional wear on the leg joints (e.g., hip, knee, and ankle). Additionally, a greater flexure of the knee during the push-off phase 625-3 causes the surrounding muscles to compensate, which may give rise to other musculoskeletal disorders.

In one non-limiting example, the external geometry of the heel support may be altered (e.g., inflated to a greater inflation state) prior to the heel strike phase 625-1. As shown in plot 660, a pressure of the heel support Ned is held at a first pressure P1 during the swing phase 625-4 of the gait cycle 660-1. In plot 665, a pressure of the longitudinal arch support Parch is held at a first pressure P4 during the swing phase 625-4.

The external geometry of the longitudinal arch support may be altered (e.g., inflated to a greater inflation state) prior to the mid-stance phase 625-2. In the plot 665, the pressure of the longitudinal arch support Parch is increased from the first pressure P4 to a second pressure P3 during the heel strike phase 625-1. In some embodiments, fluid is transferred from the heel support to the longitudinal arch support during a transfer period 670 to cause the increased pressure of the longitudinal arch support Parch.

The external geometry of the heel support may again be altered (e.g., deflated to a lesser inflation state) after the heel strike phase 625-1. In the plot 660, the pressure of the heel support Pₕₑₑₗ is decreased from the first pressure P1 to a second pressure P2 during the heel strike phase 625-1. As mentioned above, fluid may be transferred from the heel support during the transfer period 670 to cause the decreased pressure. In some embodiments, the second pressures P2, P3 are maintained during a hold period 675 of the mid-stance phase 625-2.

The external geometry of the longitudinal arch support may again be altered (e.g., deflated to a lesser inflation state) after the mid-stance phase 625-2. In the plot 665, the pressure of the longitudinal arch support arch is P decreased from the second pressure P3 to the first pressure P4 during the push-off phase 625-3. In some cases, altering the external geometry of the longitudinal arch support may include transferring fluid between the longitudinal arch support to the heel support during a transfer period 680 of the push-off phase 625-3. In some embodiments, the pressure of the heel support Pₕₑₑₗ is increased from the second pressure P2 to the first pressure P1 during the push-off phase 625-3. A pressure of the heel support Pₕₑₑₗ may be held at the first pressure P1, and/or the pressure of the longitudinal arch support Parch may be held at the first pressure P4, during a hold period 685 of the swing phase 625-4 of the gait cycle 660-2.

Thus, in the heel strike phase 625-1, the heel pressing into the inflatable bladder of the heel support generates pressure that may be relieved into the inflatable bladder of the longitudinal arch support (or another bladder(s)) through a valve. During the mid-stance phase 625-2, the valve may be held closed to hold supportive pressure in the inflatable bladder of the longitudinal arch support. In the push-off phase 625-3, the valve is opened allowing pressure to distribute to the inflatable bladder of the heel support. At the transition to the swing phase 625-4, or at a set point measured in time, pressure, or other signal parameter, the valve is closed and held until pressure is again generated by the next heel strike. The valve control may be under digital control, analog proportional control, or passive control of multiple one-way valves.

For a multiple-bladder system, each bladder may be actuated by a combination of pump (or a positive displacement pumping system) and/or one or more valves. In a powered system, a pump provides a positive pressure and a valve controls the transmission of pressurized fluid into each of the bladders simultaneously, serially, or in parallel. In some embodiments, passively actuated micro-interventions make use of shifting body weight within gait cycles (or other poses) to force fluid from one bladder to the other.

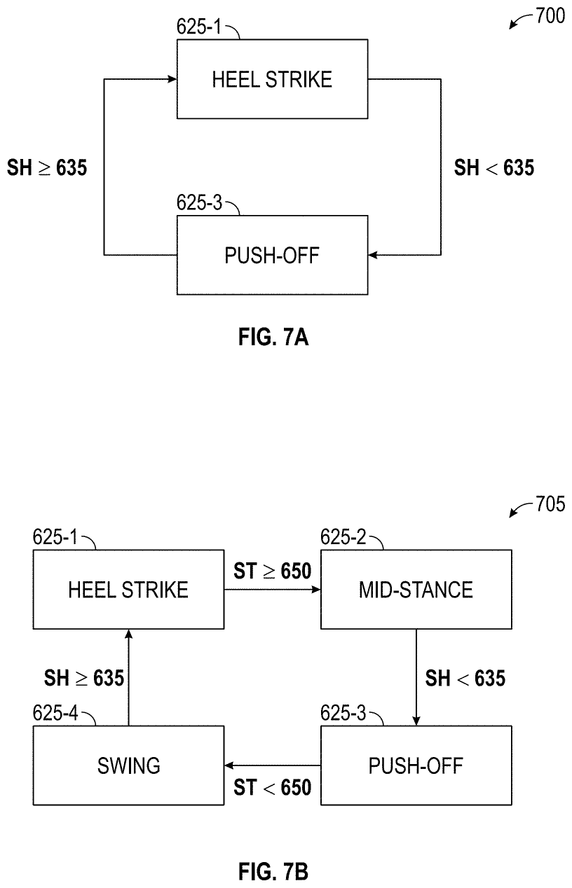

FIG. 7A illustrates a two-phase state machine 700, and FIG. 7B illustrates a four-phase state machine 705, according to one or more embodiments. The features illustrated in FIGS. 7A and 7B may be used in conjunction with other embodiments. For example, the state machines 700, 705 may be implemented into the control provided by the computer processor(s) 206, 218 of FIG. 2A. The four-phase state machine 705 may represent control using the two-sensor implementation of FIG. 6, and the two-phase state machine 700 may represent control using an alternate implementation based on a single sensor (e.g., the heel sensor of FIG. 6).

For each of the phases, the computer processor(s) 206, 218 may access threshold values and/or set point parameters for the body-worn actuatable components, and in some cases may include transition set points that specify behavior of the body-worn actuatable components during transition between the phases. In some embodiments, the threshold values and/or set point parameters may be dynamically adjusted for subsequent gait cycles based on the sensor signals.

While operation of the four-phase state machine 705 is generally discussed above with respect to FIG. 6, the two-phase state machine 700 may provide relatively simpler control based on the sensor signal 630. More specifically, the two-phase state machine 700 may rely on the first transition 640-1 and the second transition 640-2 to identify the heel strike phase 625-1 and the push-off phase 625-3. In some cases, the set point parameters for the heel strike phase 625-1 may also be applied between times t2, t3, and the set point parameters for the push-off phase 625-3 may also be applied between time t4 and a time associated with a first transition 640-1 of the next gait cycle.

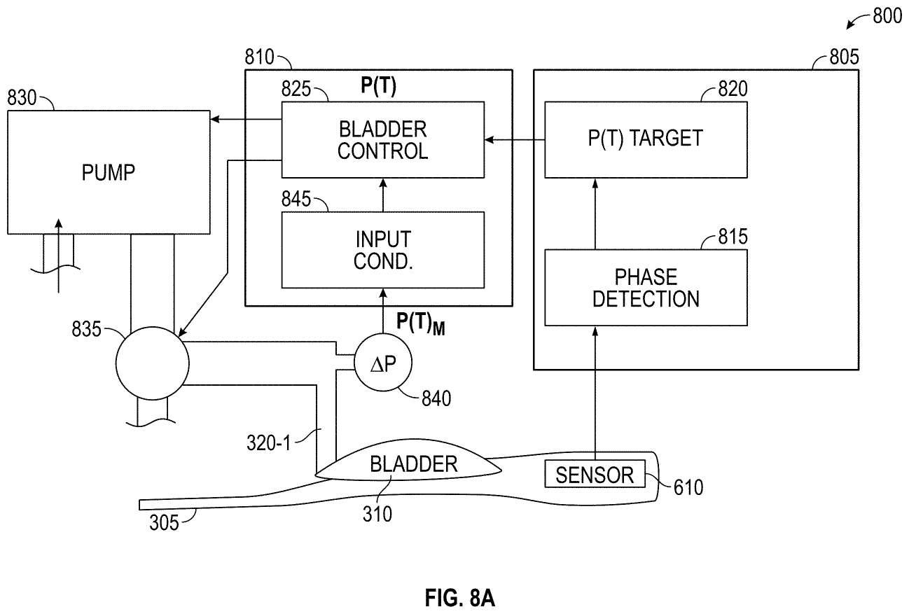

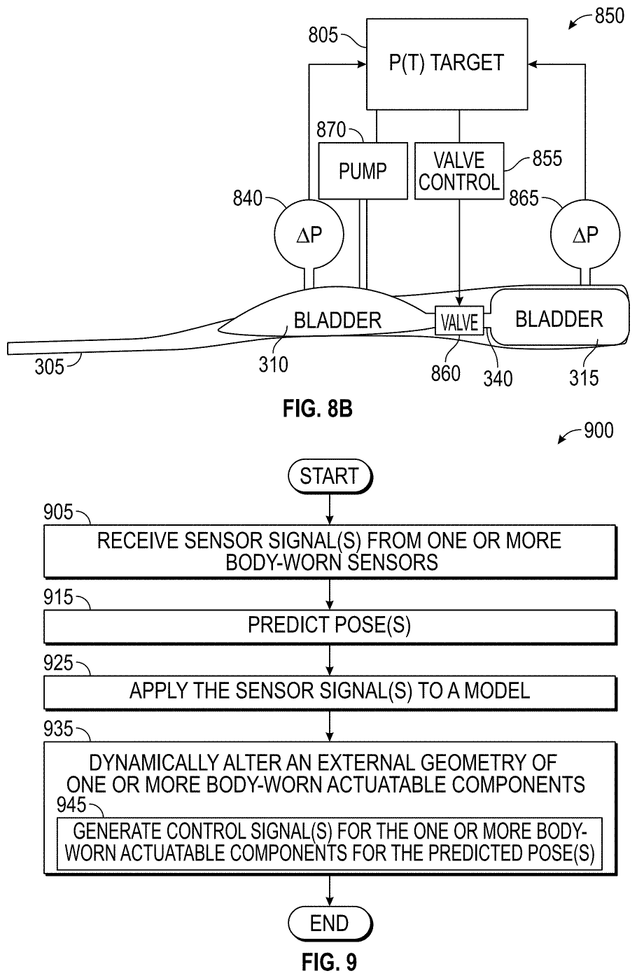

FIG. 8A is a diagram illustrating a control system 800 for an inflatable bladder in a longitudinal arch support, according to one or more embodiments. The features illustrated in FIG. 8A may be used in conjunction with other embodiments. For example, the control system 800 may be implemented into the control provided by the computer processor(s) 206, 218 of FIG. 2A.

In the control system 800, a gait controller 805 receives a sensor signal from the aft sensor 610 (e.g., a heel sensor). Using the sensor signal, a phase detection block 815 detects the phase of the gait cycle. In alternate implementations, one or more poses may be predicted by the gait controller 805 using the sensor signal. A target set point block 820 provides, based on the detected phase, a target pressure value P(t) to a control block 825 of a bladder controller 810. Although described in terms of a time-based target pressure value, alternate implementations of the control block 825 may control as a function of time, pressure, angle, or other suitable measurements.

Based on the target pressure value and a pressure measurement P(t)m acquired by a pressure sensor 840, the control block 825 transmits control signals to a pump 830 and to a valve 835 to control air pressure to the inflatable bladder of the longitudinal arch support 310. In some embodiments, a conditioning block 845 conditions the pressure measurement before being received by the control block 825. In some embodiments, the control block 825 may use “bang-bang” or on/off control, proportional (P) control, proportional-differential (PD) control, proportional-integral-differential (PID) control, or any other suitable control techniques.

In some embodiments, the pump 830 provides pressurized environmental air into the bladder system. Alternate implementations of the control system 800 contemplate other fluid sources, activation sources, and types of fluids. In some embodiments, the valve 835 is a three-port valve that directs pressurized air from the pump through the fluid port 320-1 to inflate the bladder, holds the pressure constant, or vents air from the bladder through the fluid port 320-1 to deflate the bladder.

FIG. 8B is a diagram illustrating a control system 850 for inflatable bladders in a longitudinal arch support and a heel support, according to one or more embodiments. The features illustrated in FIG. 8B may be used in conjunction with other embodiments. For example, the control system 850 may be implemented into the control provided by the computer processor(s) 206, 218 of FIG. 2A.

In the control system 850, the gait controller 805 receives a pressure measurement from a pressure sensor 840 coupled with the longitudinal arch support 310, and a pressure measurement from a pressure sensor 865 coupled with the heel support 315. The gait controller 805 provides a target pressure value to a valve controller 855 and/or to a pump 870. In some embodiments, the target pressure values are determined based on predicted pose(s) and/or the detected phase of the gait cycle. Further, alternate implementations of the control system 850 may use sensors separate from the pressure sensors 840, 865 when determining the target pressure values (e.g., the forward sensor 615 and/or the aft sensor 610 of FIG. 6).

The valve controller 855 generates one or more control signals to operate a valve 860 arranged between the longitudinal arch support 310 and the heel support 315. In this way, air (or other suitable fluid) may be permitted to flow from one bladder to the next depending on the target set points generated by the gait controller 805 for the bladder system.

In some embodiments, the parameters for the gait controller 805 may be updated by an overall system controller. Thus, the parameters for the gait controller 805 may be determined locally or remotely. For example, a cloud-based analytics system may identify a desired modification to the external geometry of the body-wearable actuatable components for different poses, during gait cycles, and so forth. The modification may then be translated to an adjustment of the target parameters of the gait controller 805. Alternatively, the target parameters may be tuned locally based on the control of the foot-wearable apparatus.

FIG. 9 is a method 900 of dynamically altering an external geometry of body-worn actuatable components, according to one or more embodiments. The method 900 may be used in conjunction with other embodiments, such as being performed by the system 200 of FIG. 2A. The method 900 begins at block 905, where sensor signals are received from one or more body-worn sensors. At block 915, one or more poses are predicted. In some embodiments, the one or more predicted poses correspond to phases of a gait cycle. At block 925, the sensor signals are applied to a model (e.g., a ML model) to determine values for dynamically altering the external geometry of the one or more body-worn actuatable components. At block 935, the values are used to dynamically alter the external geometry of the one or more body-worn actuatable components. In some embodiments, dynamically altering the external geometry comprises, at block 945, generating control signals for the one or more body-worn actuatable components for the one or more predicted poses. The method 900 ends following completion of block 935.

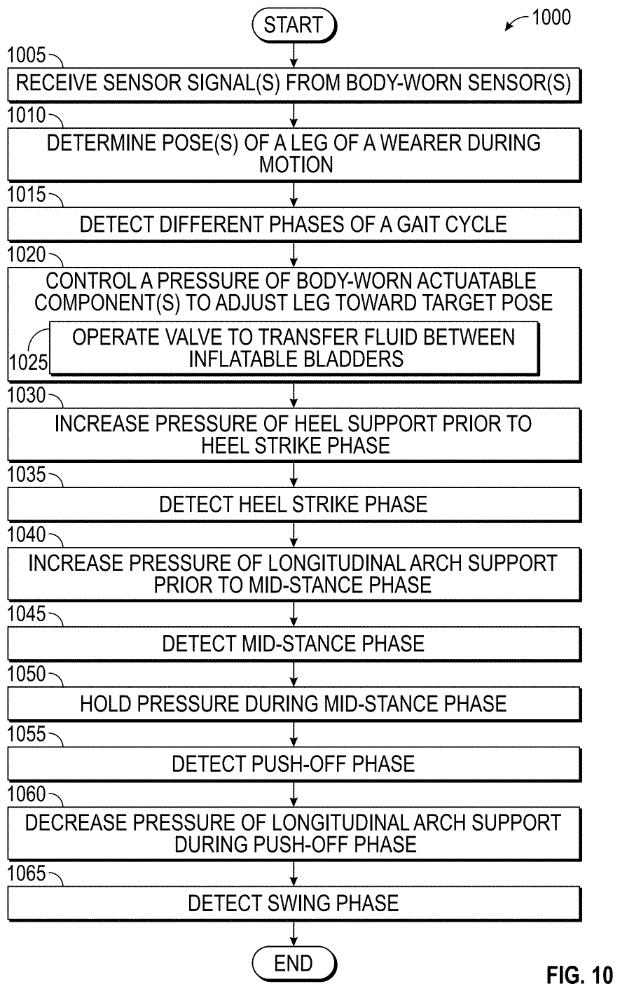

FIG. 10 is a method 1000 of dynamically altering an external geometry of foot-worn actuatable components, according to one or more embodiments. The method 1000 may be used in conjunction with other embodiments, such as being performed by the system 200 of FIG. 2A. The method 1000 begins at block 1005, where one or more sensor signals are received from one or more body-worn sensors. At block 1010, using the one or more sensor signals, one or more poses are determined of a leg of a wearer during motion. At block 1015, the different phases of a gait cycle are detected using the one or more sensor signals. At block 1020, a pressure of one or more body-worn actuatable components is controlled to adjust the leg toward a target pose. In some embodiments, controlling the pressures comprises, at block 1025, operating one or more valves to transfer fluid between inflatable bladders.

At block 1030, a pressure of a heel support is increased prior to a heel strike phase. At block 1030, a heel strike phase is detected. In some alternate implementations, the heel strike phase is predicted based on the one or more sensor signals. At block 1040, a pressure of a longitudinal arch support is increased prior to a mid-stance phase. In some embodiments, fluid is transferred from the heel support to the longitudinal arch support to increase the pressure of the longitudinal arch support. At block 1045, the mid-stance phase is detected. In some alternate implementations, the mid-stance phase is predicted based on the one or more sensor signals. At block 1050, a pressure of the longitudinal arch support and/or the heel support is held during the mid-stance phase. At block 1055, a push-off phase is detected. In some alternate implementations, the push-off phase is predicted based on the one or more sensor signals. At block 1060, a pressure of the longitudinal arch support is decreased during the push-off phase. In some embodiments, fluid is transferred from the longitudinal arch support to the heel support to decrease the pressure of the longitudinal arch support. In some embodiments, transfer of the fluid into the heel support causes the increased pressure of the heel support prior to a heel strike phase (e.g., block 1030 for a subsequent gait cycle). At block 1065, a swing phase is detected. In some alternate implementations, the swing phase is predicted based on the one or more sensor signals. The method 1000 ends following completion of block 1065.

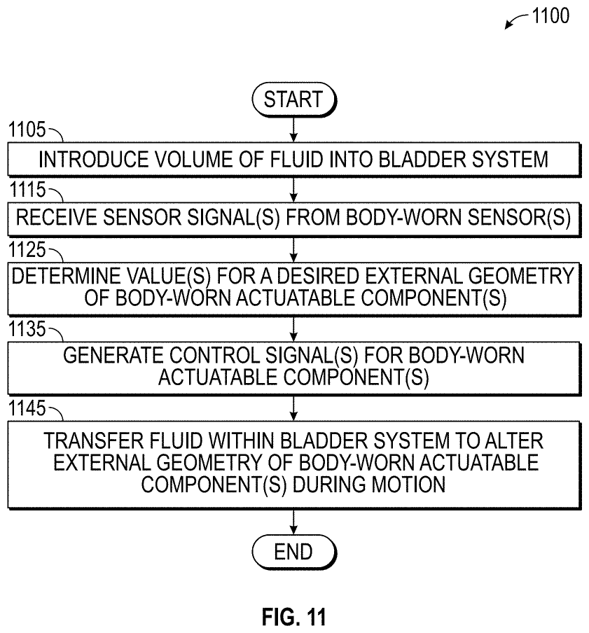

FIG. 11 is a method 1100 of controlling a bladder system to alter an external geometry of foot-worn actuatable components, according to one or more embodiments. The method 1100 may be used in conjunction with other embodiments, such as being performed by the system 200 of FIG. 2A. The method 1100 begins at block 1105, where a volume of fluid is introduced into a bladder system. In some embodiments, a manual pump, electric pump, or pressurized fluid source is used to inject fluid (e.g., environmental air) into the bladder system. At block 1115, one or more sensor signals are received from one or more body-worn sensors. At block 1125, one or more values are determined for a desired external geometry of one or more body-worn actuatable components. At block 1135, one or more control signals are generated for the one or more body-worn actuatable components. At block 1145, fluid is transferred within the bladder system to alter the external geometry of the one or more body-worn actuatable components during motion (e.g., for one or more predicted poses, for phases of a gait cycle, and so forth). The method 1100 ends following completion of block 1145.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the preceding, reference is made to embodiments presented in this disclosure. However, the scope of the present disclosure is not limited to specific described embodiments. Instead, any combination of the features and elements described herein, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the aspects, features, embodiments and advantages described herein are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

Aspects of the present invention may take the form of an entirely hardware embodiment, an entirely software embodiment (including firmware, resident software, microcode, etc.) or an embodiment combining software and hardware aspects that may all generally be referred to herein as a “circuit,” “module” or “system.”

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the FIGS. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications (e.g., a component geometry application) or related data available in the cloud. For example, the component geometry application could execute on a computing system in the cloud, receive data from body-worn actuatable components of sensor devices, and generate values for a desired external geometry using a machine-learning algorithm. Doing so allows a user to access this information from any computing system attached to a network connected to the cloud (e.g., the Internet).

While the foregoing is directed to embodiments of the present invention, other and further embodiments of the invention may be devised without departing from the basic scope thereof, and the scope thereof is determined by the claims that follow.

What is claimed is:

1. A method comprising:
receiving one or more sensor signals from one or more sensors that are worn by a wearer;
determining, based on the one or more sensor signals, one or more poses of a leg of the wearer during motion; and
controlling, based on the one or more poses, a pressure of one or more actuatable components to adjust the leg toward a target pose during the motion, wherein the one or more actuatable components are worn on one or both of the leg and a foot of the wearer.

2. The method of claim 1, wherein the one or more actuatable components comprise a knee sleeve coupled with one or more inflatable bladders.

3. The method of claim 1, wherein the one or more actuatable components comprise one or both of a longitudinal arch support and a heel support coupled with an insole.

4. The method of claim 1, wherein a valve is in fluid communication with an inflatable bladder of the one or more actuatable components, the method further comprising:
detecting, based on the one or more poses, different phases of a gait cycle of the wearer,
wherein controlling the pressure of the one or more actuatable components comprises operating the valve to transfer fluid into, or out of, the inflatable bladder during the different phases of the gait cycle.

5. The method of claim 4,
wherein a first inflatable bladder of the one or more actuatable components is in fluid communication, through the valve, with a second inflatable bladder of the one or more actuatable components, and
wherein the valve is operable to transfer fluid between the first inflatable bladder and the second inflatable bladder during the different phases of the gait cycle.

6. A method comprising:
predicting one or more poses based on one or more sensor signals from one or more body-worn sensors; and
dynamically altering an external geometry of one or more body-worn actuatable components for the predicted one or more poses.

7. The method of claim 6, further comprising:
receiving the one or more sensor signals from the one or more body-worn sensors during motion of a leg of a wearer; and
applying the one or more sensor signals to a model to determine values for dynamically altering the external geometry of the one or more body-worn actuatable components.

8. The method of claim 7, wherein the model comprises a machine learning model.

9. The method of claim 7, further comprising:
generating, using the determined values, control signals for the one or more body-worn actuatable components.

10. The method of claim 7, further comprising:
detecting, based on the one or more sensor signals, different phases of a gait cycle of the wearer; and
altering the external geometry of the one or more body-worn actuatable components according to the different phases.

11. The method of claim 6,
wherein at least one of the one or more body-worn actuatable components comprises an inflatable bladder, and
wherein dynamically altering the external geometry of the one or more body-worn actuatable components comprises altering an inflation of the inflatable bladder.

12. The method of claim 11, wherein altering the inflation of the inflatable bladder comprises:
operating a valve to transfer fluid into, or out of, the inflatable bladder during for the predicted one or more poses.

13. The method of claim 12,
wherein the one or more body-worn actuatable components comprise a first inflatable bladder in fluid communication, through the valve, with a second inflatable bladder, and wherein operating the valve transfers fluid between the first inflatable bladder and the second inflatable bladder for the predicted one or more poses.

14. A system comprising:
one or more body-wearable sensors;
one or more body-wearable, actuatable components; and
one or more computer processors configured to:
predict one or more poses based on one or more sensor signals from the one or more body-wearable sensors; and
dynamically alter an external geometry of the one or more body-wearable, actuatable components for the predicted one or more poses.

15. The system of claim 14, wherein the one or more body-wearable sensors and the one or more body-wearable, actuatable components are integrated into a body-wearable sensor device.

16. The system of claim 15, wherein the body-wearable sensor device further comprises:
a transceiver configured to communicate with a mobile computing device via a communicative link,
wherein the one or more body-wearable, actuatable components are configured to dynamically alter the external geometry of the one or more body-wearable, actuatable components based on control signals received from the mobile computing device.

17. The system of claim 16, wherein the control signals are generated by the mobile computing device based on the one or more sensor signals.

18. The system of claim 17, wherein the control signals are generated by the mobile computing device are further based on one or more sensor signals received from one or more other body-wearable sensor devices communicatively coupled with the mobile computing device.

19. The system of claim 14, wherein the one or more computer processors are further configured to:
apply the one or more sensor signals to a model to determine values for dynamically altering the external geometry of the one or more body-wearable, actuatable components for the predicted one or more poses.

20. The system of claim 19, wherein the model comprises a machine learning model.
